(19) United States
(12) Patent Application Publication
(10) Pub. No.: US 2022/0104960 A1
(43) Pub. Date: Apr. 7, 2022

(54) UNIVERSAL AND ADAPTABLE ORTHOSIS WITH PERSONALLY ADAPTABLE SENSORS

(71) Applicant: CASSIT ORTHOPEDICS LTD, Tel-Aviv (IL)

(72) Inventor: TAMAR ISH CASSIT, Tel Aviv (IL)

(73) Assignee: CASSIT ORTHOPEDICS LTD, Tel-Aviv (IL)

(21) Appl. No.: 17/424,903

(22) PCT Filed: Jan. 22, 2020
(86) PCT No.: PCT/IL2020/050089

§ 371 Date: Jul. 22, 2021

Related U.S. Application Data
(60) Provisional application No. 62/795,245, filed on Jan. 22, 2019.

(51) Int. Cl. A61F 5/058

(52) U.S. Cl. CPC A61F 5/05866

(57) ABSTRACT

A device applicable to a limb, the device may include an orthosis including or more holes extending between base surfaces of the plate; one or more sensors, at least one of the one or more sensors is adapted to be attached to at least one of the one or more holes in the orthosis; and a data collecting unit connectable to the one or more sensors and configured to receive sensors data from the one or more sensors.

### FIELD OF THE INVENTION

The present invention relates to the field of orthoses, and more particularly, to orthoses capable of monitoring a healing, preservation, protection or rehabilitation process of a limb.

### BACKGROUND OF THE INVENTION

Orthopedic limb injuries (e.g., such as injured bones, muscles, nerves, etc.) may require applying orthosis to the limb. It may be desirable to monitor one or more parameters related to a healing process of the limb, as incomplete healing process of the injured limb may, for example, result in a permanent damage to the limb. Continuous monitoring of the injured limb may, for example, contribute to completion of the healing process thereof.

### SUMMARY OF THE INVENTION

Some aspects of the present invention may provide a device applicable to a limb, the device may include: an orthosis including one or more holes extending between base surfaces of the orthosis; one or more sensors, at least one of the one or more sensors is adapted to be attachable to at least one of the one or more holes in the orthosis; and a data collecting unit connectable to the one or more sensors and configured to receive sensors data from the one or more sensors.

In some embodiments, each of the one or more sensors is selected from a group consisting of: motion sensors, muscle sensors, blood flow sensors, distance sensors, IR Proximity Sensor, force sensors, touch sensors, tilt switch sensors, angle sensors, sound sensors, accelerometer sensors, flex sensors, magnet sensors, RFID sensors, temperature sensors, light sensors, vibration sensors, pressure sensors, ECG sensors, EEG sensors and PPG sensors.

In some embodiments, the orthosis may include one or more plates formable into an orthotic member under heating.

In some embodiments, at least one of the one or more sensors is adapted to be placed within at least one of the one or more holes in the orthosis.

In some embodiments, at least one of the one or more sensors may include: a sensing unit; a sensor connector adapted to be placed within at least one of the one or more holes in the orthosis; and a sensor wire connecting the sensing unit to the sensor connector.

In some embodiments, the sensor connector may include the data collecting unit.

In some embodiments, the device may further include a pad accommodating at least one of the one or more sensors and a sensor wire or the data collecting unit.

In some embodiments, relative locations of the at least one sensor and a sensor wire or the data collecting unit in the pad are set to match locations of at least some of the holes in the orthosis.

In some embodiments, the pad may include one or more pad connectors each adapted to be placed within at least one of the one or more holes in the orthosis.

In some embodiments, the device may further include including one or more fasteners removably connectable to the orthosis and adapted to fasten the orthosis to the limb

In some embodiments, at least one of the one or more sensors is attachable to at least one of the one or more fasteners.

Another aspect of the present invention may provide a system for monitoring a healing processes of a limb, the system may include: a device applicable to a limb as described above; and a computing device connectable to the data collecting unit of the device and configured to receive the sensors data from the data collecting unit thereof.

In some embodiments, the computing device is configured to: send at least a part of the sensors data to a remote computing device; and receive, from the remote computing device, at least one of one or more feedbacks and one or more recommendations related to the healing process of the limb.

In some embodiments, the computing device is configured to determine at least one of one or more feedbacks and one or more recommendations related to the healing process of the limb based on at least a part of the sensors data and a reference dataset.

In some embodiments, the system may further include a user interface, the computing device is configured to present, on the user interface, at least one of the one or more feedbacks and the one or more recommendations.

In some embodiments, the system may further include a communication unit, the computing device is configured to send, via the communication unit, at least one of the one or more feedbacks and the one or more recommendations to an authorized third party.

In some embodiments, the system may further include a second computing device configured to: receive, from the computing device, at least a part of the sensors data; determine at least one of one or more feedbacks and one or more recommendations related to the healing process of the limb based on the received sensors data and a reference dataset; and send at least one of the one or more feedbacks and the one or more recommendations to the computing device.

In some embodiments, the device may include one or more three-dimensional accelerometers, and wherein the second computing device may be configured to: determine, based on readings of the one or more accelerometers, information concerning a motility of the device, and determine at least one of the one or more feedbacks and the one or more recommendations based on the information concerning the motility of the device.

In some embodiments, the second computing device is configured to: associate the sensors data with at least one of the determined one or more feedbacks and the determined one or more recommendations; and update the reference dataset based on the sensors data associated with at least one of the determined one or more feedbacks and the determined one or more recommendations.

In some embodiments, the system may further include a system database, the second computing device is configured to build a system reference dataset based on sensors data collected from multiple devices for supporting a limb and at least one of the determined one or more feedbacks and the determined one or more recommendations associated therewith.

In some embodiments, at least one of the one or more feedbacks and the one or more recommendations are generated based on the system reference dataset.

Another aspect of the present invention may provide a method for applying a device to a limb and monitoring a healing process of the limb, the method may include: providing a device applicable to a limb, the device may include: an orthosis including one or more holes extending between base surfaces of the orthosis, one or more sensors, and a data collecting unit; attaching at least one of the one or more sensors to at least one of the one or more holes in the orthosis; fastening the orthosis to the limb; receiving, by the data collecting unit, sensors data from the at least one of the one or more sensors; sending, by the data collecting unit, the sensors data to a computing device; and generating, by at least one of the computing device and a remote computing device, at least one of one or more feedback and one or more recommendations related to the healing process of the limb based on at least a part of the sensors data and a reference dataset.

Some embodiments may include selecting each of the one or more sensors from a group consisting of: motion sensors, muscle sensors, blood flow sensors, distance sensors, force sensors, touch sensors, tilt switch sensors, angle sensors, sound sensors, accelerometer sensors, flex sensors, magnet sensors, RFID sensors, temperature sensors, light sensors, vibration sensors, pressure sensors, ECG sensors, EEG sensors and PPG sensors.

If the device includes one or more three-dimensional accelerometers, some embodiments may include: determining, based on readings of the one or more accelerometers, information concerning a motility of the device, and determining at least one of the one or more feedbacks and the one or more recommendations based on the information concerning the motility of the device.

Some embodiments may include forming the orthosis from a plate under heating.

Some embodiments may include attaching at least one of the one or more sensors to at least one of one or more fasteners that fasten the orthosis to the limb.

Some embodiments may include presenting at least one of the one or more feedbacks and the one or more recommendations to a user.

Some embodiments may include providing at least one of the one or more feedbacks and the one or more recommendations using at least one of visual, audio and vibrational notification.

Some embodiments may include sending at least one of the one or more feedbacks and the one or more recommendations to an authorized third party.

Some embodiments may include: associating the sensors data with at least one of the determined one or more feedbacks and the determined one or more recommendations; and updating the reference dataset based on the sensors data associated with at least one of the determined one or more feedbacks and the determined one or more recommendations.

Some embodiments may include building a second reference dataset based on sensors data collected from multiple devices for supporting a limb and at least one of the determined one or more feedbacks and the determined one or more recommendations associated therewith.

Some embodiments may include generating at least one of the one or more feedback and the one or more recommendations based on the second reference dataset.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIGS. 1A, 1B, 1C, 1D and 1E are schematic illustrations of a device applicable to a limb, according to some embodiments of the invention;

FIG. 1F is a schematic illustration of a device applicable to a limb and of one embodiment of a sensor for the device, according to some embodiments of the invention.

FIGS. 2A and 2B are schematic illustrations of one embodiment of a device applicable to a limb and of the device applied to the limb, respectively, according to some embodiments of the invention;

FIGS. 3A and 3B are schematic block diagrams of a system for monitoring a healing process of a limb, according to some embodiments of the invention; and

FIG. 4 is a flowchart of a method of applying a device to a limb and monitoring a healing process of the limb, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Any of the disclosed modules or units can be at least partially implemented by a computer processor.

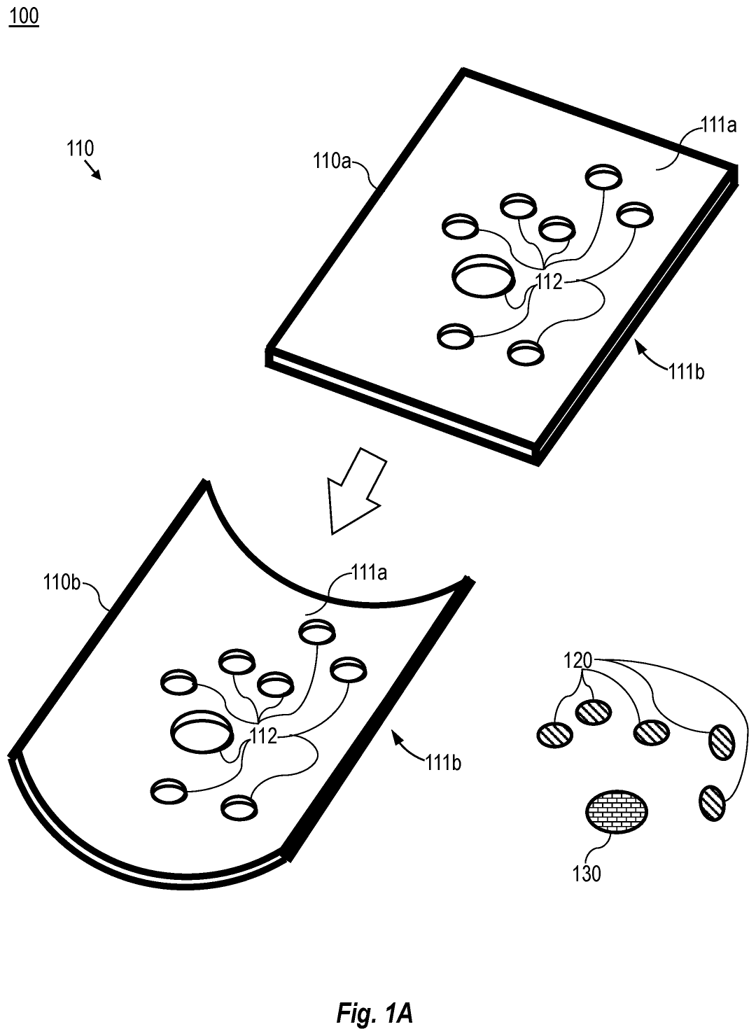

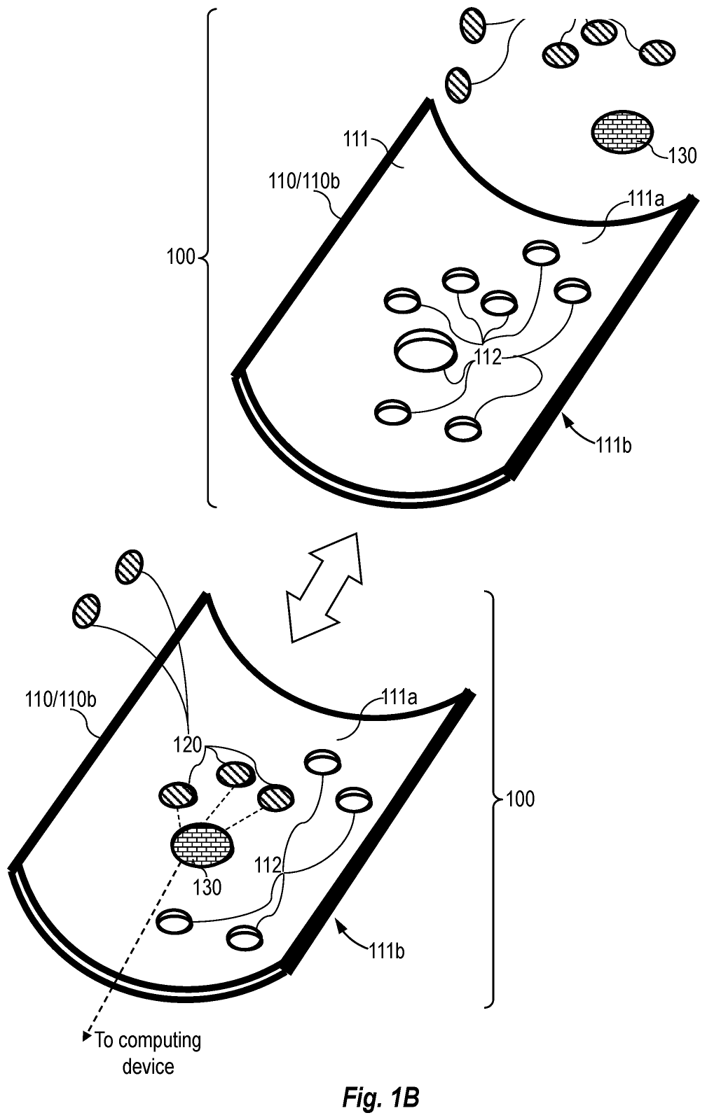

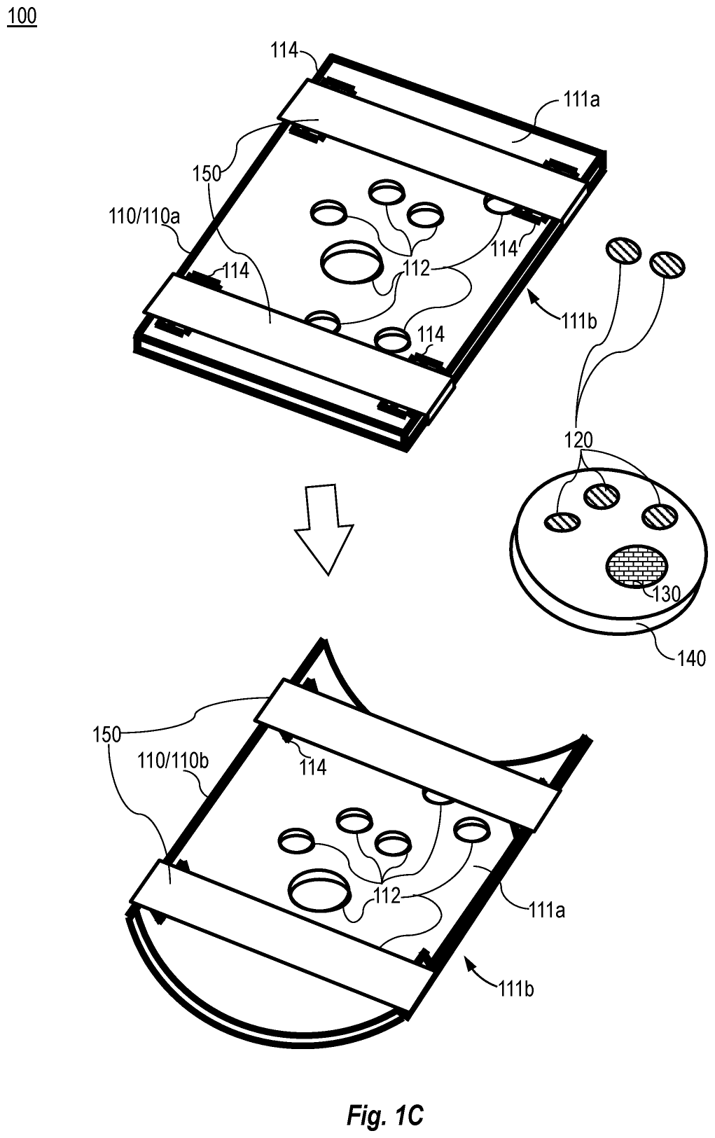

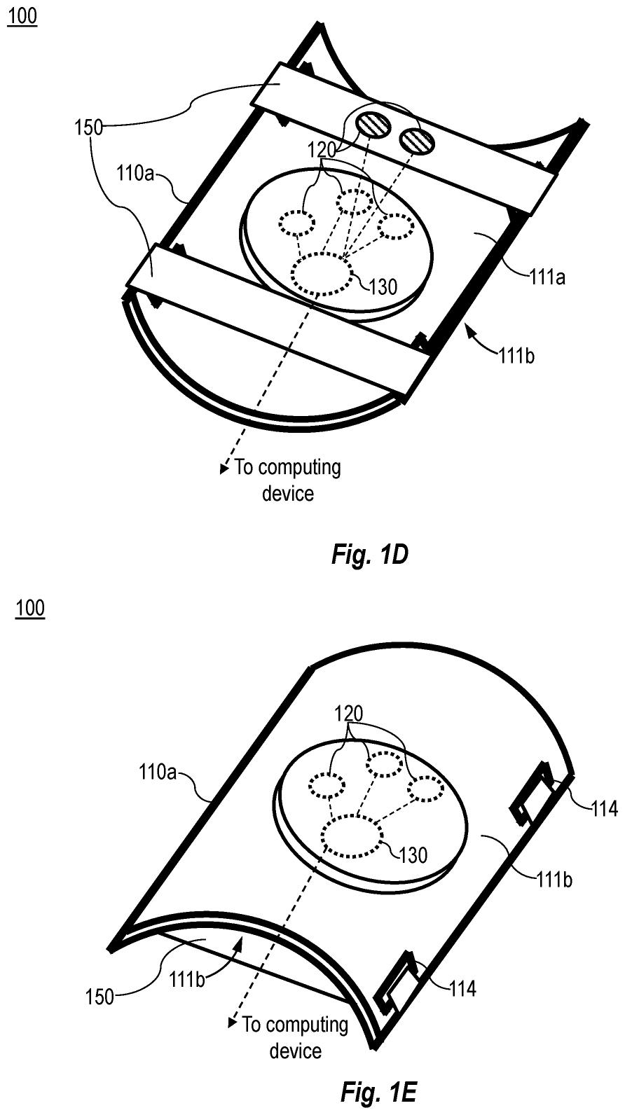

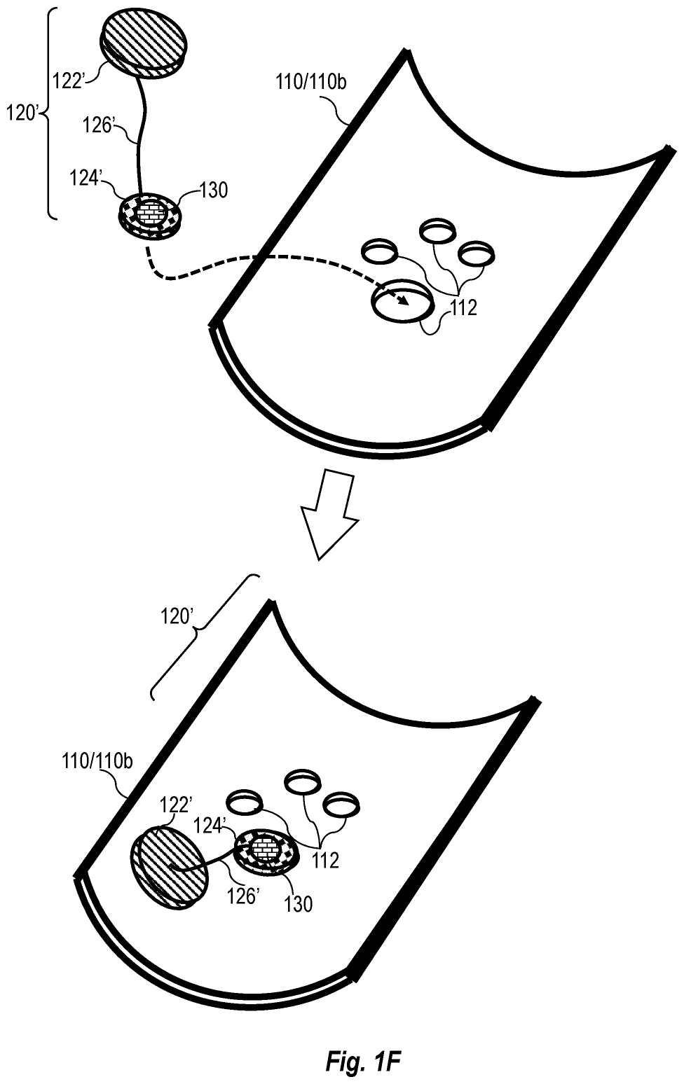

Reference is now made to FIGS. 1A, 1B, 1C, 1D, 1E and 1F, which are schematic illustrations of a device 100 applicable to a limb, according to some embodiments of the invention.

Device 100 may be applicable to an entire limb (e.g., an arm or a leg) or a portion thereof (e.g., a finger, a joint, a segment of bone, etc.). Device 100 may be applicable to a human limb or an animal limb. Device 100 may support, immobilize or improve a function of a limb. Device 100 may enable monitoring a healing process of a limb.

According to some embodiments, device 100 may include an orthosis 110, one or more sensors 120 and a data collection unit 130 (e.g., as shown in FIGS. 1A, 1B and 1C).

Orthosis 110 may be applicable to a limb or a portion thereof and configured to stabilize or immobilize the limb, improve alignment, prevent deformities, protect against injury, or assist with motion or function of the limb.

In some embodiments, orthosis 110 may be pre-shaped into a desired shape. For example, the shape and dimension may depend on a type of orthosis 110 (e.g., a splint, a cast, a prothesis, etc.), a type of a limb (leg, arm, finger, etc.), a type of a limb injury or disorder (e.g., bone injury, muscle injury, nerve injury, cerebral palsy disorder, etc.) and/or subject-related parameters (e.g., age, height, etc.)

In some other embodiments, orthosis 110 may include a plate 110a that may be formed into an orthotic member 110b (e.g., as shown in FIGS. 1A and 1C). For example, plate 110a may be made of a thermoplastic polymer. When the thermoplastic polymer is heat to a temperature above a glass transition temperature thereof, the thermoplastic polymer softens and allows shaping plate 110a into orthotic member 110b of a desired spatial configuration. When the thermoplastic polymer is cooled to a temperature below the glass transition temperature, the thermoplastic polymer hardens and fixates the spatial configuration of orthotic member 110b. When formed, orthotic member 110b may support, immobilize or improve a function of a limb or at least a portion thereof.

Plate 110a may have different shapes and dimensions, depending on a type of orthotic member 110b to be formed (e.g., a splint, a cast, a prothesis, etc.), a type of a limb (leg, arm, finger, etc.), a type of a limb injury or disorder (e.g., bone injury, muscle injury, nerve injury, cerebral palsy disorder, etc.) and/or subject-related parameters (e.g., age, height, etc.).

Orthosis 110, or plate 110a formable into orthotic member 110b, may include one or more holes 112 extending through orthosis 110 between base surfaces 111a, 111b thereof (e.g., as shown in FIGS. 1A, 1B and 1C). In some embodiments, orthosis 110 may include a plurality of holes 112. Different holes 112 may have different shapes and dimensions. At least one hole of holes 112 may be adapted to, and optionally removably, receive at least one of sensors 120 and/or data collecting unit 130, or portions thereof (e.g., as shown in FIG. 1B). At least one hole of holes 112 that is not occupied by sensors 120 and/or data collecting unit 130 may enable aeration of a limb supported by orthosis 110. Holes 112 may also reduce a weight of orthosis 110.

In some embodiments, orthosis 110 may be made of a transparent material.

In some embodiments, at least one of sensors 120 may be adapted to be attachable, and optionally removably attachable, to at least one of holes 112. In some embodiments, at least one of sensors 120 may be adapted to be placed, and optionally removably placed, within at least one of holes 112. In some embodiments, at least one of sensors 120 may be adapted to be attached directly to a limb. Sensors 120 may be adapted to measure different parameters related to, for example, a healing process of a limb. For example, each of sensors 120 may be selected from a group consisting of: motion sensors, muscle sensors, blood flow sensors, distance sensors, force sensors, touch sensors, tilt switch sensors, angle sensors, sound sensors, accelerometer sensors, flex sensors, magnet sensors, RFID sensors, temperature sensors, light sensors, vibration sensors, pressure sensors, ECG sensors, EEG sensors and PPG sensors. It is noted that other sensors may be also used.

Data collecting unit 130 may be in communication (e.g., wired or wireless communication) with at least some of sensors 120 and may be configured to receive sensors data from sensors 120. In some embodiments, data collecting unit 130 may be a connector configured to transfer sensors data to an external computing device. In various embodiments, data collecting unit 130 (or at least a portion thereof) may be adapted to be placed, and optionally removably placed, within at least one of holes 112 in orthosis 110, or data collecting unit 130 may be attachable, or optionally removably attachable, to one of surfaces of orthosis 110 or directly to a limb. Data collecting unit 130 may be connectable (e.g., by wire or wirelessly) to a computing device and configured to send at least a part of the sensors data from sensors 120 to the computing device for further processing (e.g., as shown in FIGS. 1B, 1D and 1E).

In some embodiments, at least some of holes 112 in orthosis 110 may be arranged according to a predefined ordered array. For example, locations of at least some holes of holes 112 in orthosis 110 may be positioned at predefined locations based on at least one of a type of orthosis 110 (e.g., a splint, a cast, a prothesis, etc.), a type of a limb (leg, arm, finger, etc.), a type of a limb injury or disorder (e.g., bone injury, muscle injury, nerve injury, cerebral palsy disorder, etc.) and/or subject-related parameters (e.g., age, height, etc.). At least some holes of holes 112 may be randomly arranged in orthosis 110. Selection of sensors 120 and location of sensors 220 in orthosis 110 may be also based on at least one of type of the splint, type of the limb, type of the limb injury or disorder and/or the subject-related parameters.

For example, for a functional/dynamic limb with a splint, a motion sensor may be used to measure a motility of the limb and a tension sensor may be used to measure a tension of muscles in the limb. The sensors may be located in regions where the muscles are relatively close to a skin of the limb. In another example, for a rest splint (e.g., for fixing a bone fracture where movement of the limb is not expected and/or not recommended), a blood flow sensor may be used to measure a blood flow to the limb. The sensor may be located in regions in which the blood flow may be monitored. In another example, for a nerve rehabilitation splint, a nerve activity the sensor may be used to measure a nerve activity in the limb and a motion sensor may be used to measure a motility of the limb. The sensors may be located in regions where the nerves are relatively close to a skin of the limb.

In some embodiments, orthosis 110 may include a plurality of holes 112 of different shapes and dimensions and arranged in different regions of orthosis 110 to provide a flexibility in positioning of different sensors 120 in different regions of orthosis 110.

In some embodiments, device 100 may include a flexible pad 140 (e.g., as shown in FIGS. 1C, 1D and 1E). For example, flexible pad 140 may be made of silicone or any other flexible material. Pad 140 may accommodate at least some of sensors 120 and/or data collecting unit 130 (e.g., as shown in FIGS. 1C, 1D and 1E). In some embodiments, pad 140 may include electronic circuit(s) that enable communication between sensors 120 and data collecting unit 130. Relative locations of sensors 120 and/or data collecting unit 130 in flexible pad 140 may be set to match relative locations of at least some of holes 112 in orthosis 110 to enable placing sensors 120 and/or data collecting unit 130 within respective holes. In some embodiments, flexible pad 140 may be placed on a concave surface of splint 110b (e.g., surface 111a as shown in example of FIG. 1D) or a convex surface of splint 110b (e.g., surface 111b as shown in example of FIG. 1E).

In some embodiments, device 100 may include one or more fasteners 150 (e.g., as shown in FIGS. 1C, 1D and 1E). Fastener(s) 150 may be removably connectable to orthosis 110. For example, fastener(s) 150 may include hook and loop connectors. Orthosis 110 may include one or more pairs of slits 114 that enable connection of fastener(s) 150 to orthosis 110. Fastener(s) 150 may fasten orthosis 110 to a limb. In some embodiments fasteners 150 may be made of flexible hook-and-pile fasteners straps.

In some embodiments, one or more of sensors 120 may be attachable to one or more of fastener(s) 150 (e.g., as shown in FIG. 1C). The sensor(s) thereof may be in communication (e.g., wired or wireless) with existing data collecting unit 130, or device 100 may include a second data collection unit to collect data from the sensor(s).

Reference is now made to FIG. 1F, which is a schematic illustration of a device 100 applicable to a limb and of one embodiment of a sensor 120′ thereof, according to some embodiments of the invention.

In some embodiments, sensor 120′ may include a sensing unit 122′ and a sensor connector 124′. Sensing unit 122′ may be configured to measure one or more parameters related to, for example, a healing process of a limb. Sensor connector 124′ may be adapted to be attached, or removably attached, to at least one of holes 112 in orthosis 110. For example, sensor connector 124′ may be optionally removably, placed within at least one of holes 112. In some embodiments, sensor 120′ may include a sensor wire 126′ connecting sensing unit 122′ to sensor connector 124′. Sensor wire 126′ may enable locating and/or relocating sensing unit 122′ of sensor 120′ in a predetermined radius from sensor connector 124′ and hole 112 accommodating sensor connector 124′. In some embodiments, sensor connector 124′ may include data collecting unit 130.

In some embodiments, at least one of sensors 120 described above with respect to FIGS. 1A, 1B, 1C, 1D, and 1E may be similar to sensor 120′ described above with respect to FIG. 1F.

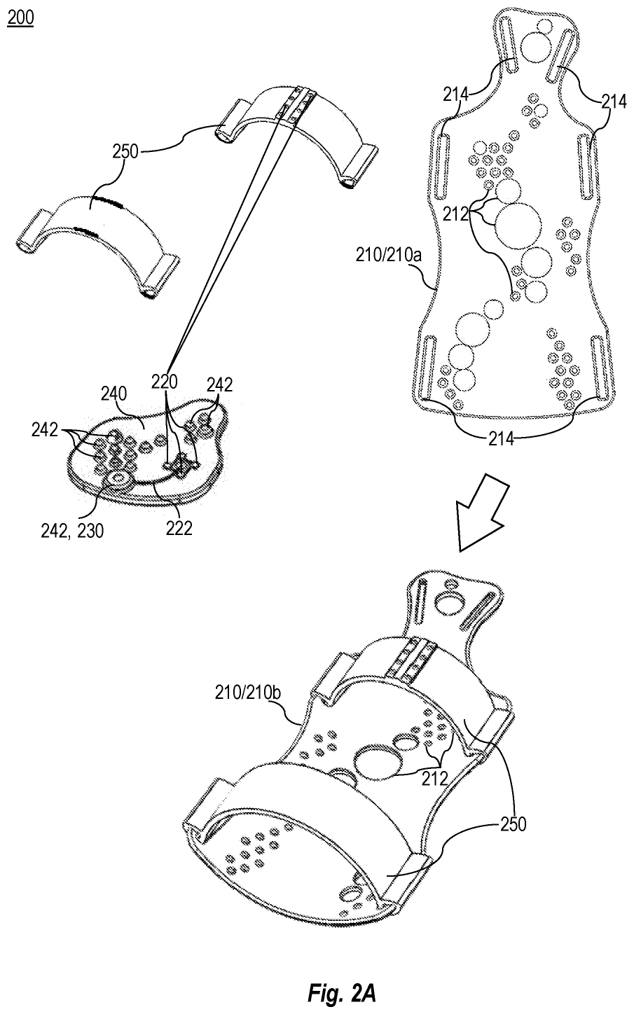

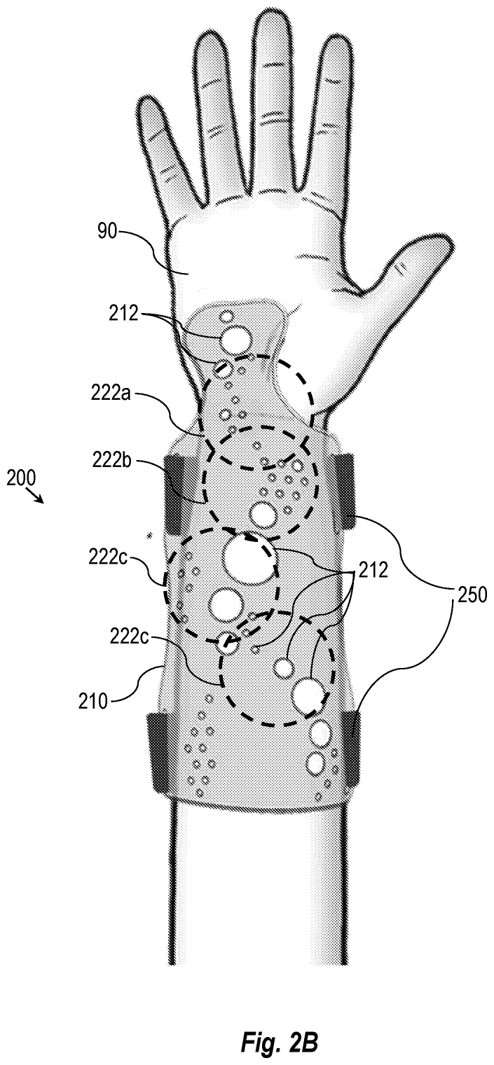

Reference is now made to FIGS. 2A and 2B, which are schematic illustrations of one embodiment of a device 200 applicable to a limb 90 and of device 200 applied to limb 90, respectively, according to some embodiments of the invention.

Device 200 may be similar to device 100 described above with respect to FIGS. 1A, 1B, 1C, 1D, 1E and 1F. Device 200 may include an orthosis 210 (or a plate 210a formable into orthotic member 210b) including holes 212, one or more sensors 220 and a data collecting unit 230.

In some embodiments, device 200 may include a flexible pad 240. Flexible pad 240 may accommodate at least some of sensors 220 and/or data collecting unit 230. In some other embodiments, data collecting unit 230 may be attachable, or optionally removably attachable, to one of surfaces of orthosis 210 or directly to a limb. In some embodiments, flexible pad 240 may include one or more pad connectors 242 adapted to be optionally removably, placed within at least one of holes 212 to thereby attach flexible pad 240 to orthosis 210.

In some embodiments, at least one of sensors 220 may be attached to at least one of pad connectors 242 using at least one sensor wire 222. In some embodiments, pad connector(s) 242 that are attached to at least one of sensors 220 may include data collecting unit 230. In some other embodiments, data collecting unit 230 may be attachable, or optionally removably attachable, to one of surfaces of orthosis 110 or directly to a limb. In some embodiments, data collecting unit 230 may be a connector configured to transfer sensors data to an external computing device.

In some other embodiments, at least one of sensors 220 may be adapted to be attachable, and optionally removably attachable, to at least one of holes 212. In some other embodiments, at least one of sensors 220 may be adapted to be places, and optionally removably placed, within at least one of holes 212. In some other embodiments, at least one of sensors 220 may be adapted to be attached directly to a limb.

In some embodiments, device 200 may include one or more fasteners 250 (e.g., similar to fastener(s) 150 described above with respect to FIGS. 1C, 1D and 1E). Fastener(s) 250 may be removably connectable to orthosis 210, or orthotic member 210b formed from plate 210a, using two or more slits 214. Fastener(s) 250 may fasten orthosis 210 to limb 90 (e.g., as shown in FIG. 2B). In some embodiments, at least one of sensors 220 may be connectable to at least one of fastener(s) 250 (e.g., as shown in FIG. 2A).

FIG. 2B depicts examples for possible locations 222a-220d of sensors 220 in orthosis 210. It is noted that sensors 220, data collecting unit 230 and pad 240 are not shown in FIG. 2B for sake of clarity only. In general, locations 222a-220d of sensors 220 in orthosis 210 may be made at predefined locations based on a type of orthosis 210 (e.g., a splint, a cast, a prothesis, etc.), a type of a limb (leg, arm, finger, etc.), a type of a limb injury or disorder (e.g., bone injury, muscle injury, nerve injury, cerebral palsy disorder, etc.) and/or subject-related parameters (e.g., age, height, etc.), for example as described above with respect to FIGS. 1A, 1B, 1C, 1D, 1E and 1F.

In some embodiments, orthosis 210 may include a plurality of holes 212 of different shapes and dimensions and arranged in different regions of orthosis 210 to provide a flexibility is positioning of different sensors 220 in different regions of orthosis 210 (e.g., as described above with respect to FIGS. 1A, 1B, 1C, 1D, 1E and 1F).

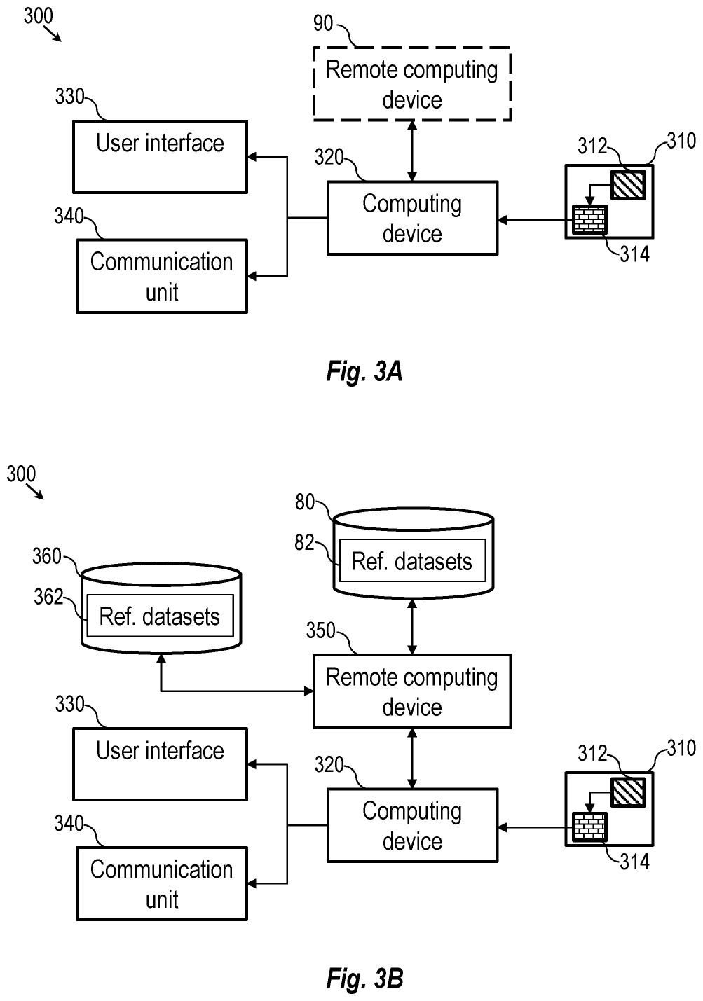

Reference is now made to FIGS. 3A and 3B, which are schematic block diagrams of a system 300 for monitoring a healing process of a limb, according to some embodiments of the invention.

According to some embodiments, system 300 may include a device 310 applicable to a limb, a computing device 320, a user interface 330 and a communication unit 340.

Device 310 may be similar to device 100 described above with respect to FIGS. 1A, 1B, 1C, 1D, 1E and 1F and/or to device 200 described above with respect to FIGS. 2A and 2B. For example, device 310 may include an orthosis, sensors 312 and data collecting unit 314. The orthosis may support, immobilize or improve a function of a limb of a user, sensors 312 may measure different parameters related to a healing process of the limb and send sensors data to data collecting unit 314 (e.g., as described above with respect to FIGS. 1A, 1B, 1C, 1D, 1E and 1F).

Computing device 320 may be connectable (e.g., wired or wirelessly) to data collecting unit 316 of device 300. Computing device 320 may receive sensors' data from data collecting unit 316 of device 300. In some embodiments, computing device 320 may send at least a part of the sensors' data to a remote computing device 90. Remote computing device 90 may analyze the sensors data received from computing device 320, generate at least one of one or more feedbacks and one or more recommendations related to a healing process of a limb based on the sensors data, and send at least one of the feedback and the one or more recommendations to computing device 320. In some embodiments, at least a part of the analysis may be performed on computing device 320. The feedback(s) may, for example, include a positive feedback indicating a positive healing process of the limb and may include encouraging comments aimed to urge the user to keep on in the healing path. The recommendation(s) may, for example, include further limb treatment operations or exercising recommendations, repositioning of sensors 312 in device 310 in order to extract data from different locations, etc.

In some embodiments, computing device 320 may present, on user interface 330, at least one of the feedbacks and/or one or more recommendations to a user. In some other embodiments, user interface 330 may provide at least one of the feedbacks and/or one or more recommendations using at least one of visual, audio and vibrational notification. In some embodiments, computing device 320 may send, using communication unit 340, at least one of the feedbacks and the one or more recommendations to an authorized third party (e.g., user's physician, etc.).

In some embodiments, computing device 320, user interface 330 and communication unit 340 may be implemented on a single device (e.g., a personal computer, a smartphone, etc.).

In some embodiments, system 300 may include a remote computing device 350 (e.g., as shown in FIG. 3B). Remote computing device 350 may be in communication (e.g., wired or wireless) with computing device 320 and a database 80. Database 80 may, for example, include a reference dataset 82. Reference dataset 82 may include a plurality of predefined sets of sensors data associated with healing patterns of different limbs and indicative of a healing process of the limbs.

Remote computing device 350 may receive at least a portion of sensors data from computing device 320. Remote computing device 350 may determine, based on the received sensors data and reference dataset 82, at least one of the one or more feedbacks and the one or more recommendations related to related to the healing process of the limb. For example, remote computing device 350 may utilize artificial intelligence (AI) methods to determine the feedback(s) and the recommendations(s).

In some embodiments, sensors 312 of device 300 may include one or more three-dimensional accelerometers attachable to the orthosis of device 300 in one or more predetermined locations. Remote computing device 350 may determine, based on readings from the accelerometer(s), information concerning a motility of device 300 (and thus of the limb). The motility may, for example, include a spatial motility of device 300 (and thus of the limb) and relative motility of the accelerometer(s) with respect to each other (and thus bending, torsion, etc. of device 300 and the limb). Remote computing device 350 may determine at least one of the one or more feedbacks and the one or more recommendations based on the information concerning the motility of device 300.

In some embodiments, remote computing device 350 may associate the received sensors data with at least one of the determined feedback(s) and the determined recommendation(s). Remote computing device 350 may update reference dataset 82 based on the sensors data associated with the at least one of the determined feedback(s) and the determined recommendation(s).

In some embodiments, system 300 may include a system database 360. Remote computing device 350 may build a system reference dataset 362 based on sensors data collected from multiple devices 310 and at least one of the determined one or more feedbacks and the determined one or more recommendations associated therewith. The data that builds system reference dataset 362 may be collected by remote computing device 350 for a specified period of time.

In some embodiments, e.g., once system reference dataset 362 includes enough amount of data, remote computing device 350 may determine at least one of the feedback(s) and recommendation(s) related to the healing process of the limb based on system reference dataset 362.

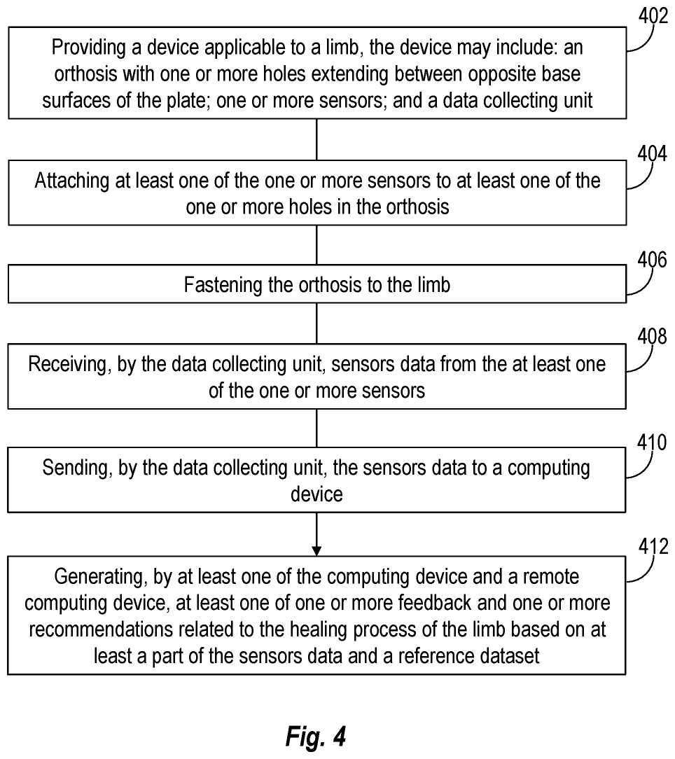

Reference is now made to FIG. 4, which is a flowchart of a method for applying a device to a limb and monitoring a healing process of the limb, according to some embodiments of the invention.

The method may be implemented by a device for supporting a limb (e.g., device 100 described above with respect to FIGS. 1A, 1B, 1C, 1D, 1E and 1F or device 200 described above with respect to FIGS. 2A and 2B) and a system for monitoring a healing process of a limb (e.g., system 300 described above with respect to FIGS. 3A and 3B), which may be configured to implement the method. It is noted that the method is not limited to the flowcharts illustrated in FIG. 4 and to the corresponding description. For example, in various embodiments, the method needs not move through each illustrated box or stage, or in exactly the same order as illustrated and described.

Some embodiments may include providing a device applicable a limb, the device may include: an orthosis including one or more holes extending between base surfaces of the orthosis; one or more sensors; and a data collecting unit (stage 402). For example, device 100 described above with respect to FIGS. 1A, 1B, 1C, 1D, 1E and 1F or device 20 described above with respect to FIGS. 2A and 2B.

Some embodiments may include selecting each of the one or more sensors from a group consisting of: motion sensors, muscle sensors, blood flow sensors, distance sensors, force sensors, touch sensors, tilt switch sensors, angle sensors, sound sensors, accelerometer sensors, flex sensors, magnet sensors, RFID sensors, temperature sensors, light sensors, vibration sensors, pressure sensors, ECG sensors, EEG sensors and PPG sensors. For example, as described above with respect to FIGS. 1A, 1B, 1C, 1D, 1E and 1F.

Some embodiments may include forming the orthosis from a plate. For example, as described above with respect to FIGS. 1A, 1B, 1C, 1D, 1E and 1F.

Some embodiments may include forming the orthosis from the plate under heating. For example, as described above with respect to FIGS. 1A, 1B, 1C, 1D, 1E and 1F.

Some embodiments may include attaching at least one of the one or more sensors to at least one of the one or more holes in the orthosis (stage 404). For example, as described above with respect to FIGS. 1A, 1B, 1C, 1D, 1E and 1F.

Some embodiments may include attaching at least one of the one or more sensors to at least one of one or more fasteners that fasten the splint to the limb. For example, as described above with respect to FIGS. 1A, 1B, 1C, 1D, 1E and 1F and FIGS. 2A and 2B.

Some embodiments may include fastening the orthosis to the limb (stage 406). For example, as described above with respect to FIGS. 1A, 1B, 1C, 1D, 1E and 1F.

Some embodiments may include receiving, by the data collecting unit, sensors data from the at least one of the one or more sensors (stage 408). For example, as described above with respect to FIGS. 1A, 1B, 1C, 1D, 1E and 1F.

Some embodiments may include sending, by the data collecting unit, the sensors data to a computing device (stage 410). For example, as described above with respect to FIGS. 1A, 1B, 1C, 1D, 1E and 1F.

Some embodiments may include generating, by at least one of the computing device and a remote computing device, at least one of one or more feedback and one or more recommendations related to the healing process of the limb based on at least a part of the sensors data and a reference dataset (stage 412). For example, as described above with respect to FIGS. 1A, 1B, 1C, 1D, 1E and 1F and FIGS. 3A and 3B.

If the one or more sensors include one or more three-dimensional accelerometers, some embodiments may include determining, based on readings of the one or more accelerometers, information concerning a motility of the device and determining at least one of the one or more feedbacks and the one or more recommendations based on the information concerning the motility of the device.

Some embodiments may include presenting at least one of the one or more feedbacks and the one or more recommendations to a user. For example, as described above with respect to FIGS. 3A and 3B. Some embodiments may include providing at least one of the one or more feedbacks and the one or more recommendations using at least one of visual, audio and vibrational notification. For example, as described above with respect to FIGS. 3A and 3B.

Some embodiments may include sending at least one of the one or more feedbacks and the one or more recommendations to an authorized third party. For example, as described above with respect to FIGS. 3A and 3B.

Some embodiments may include associating the sensors data with at least one of the determined one or more feedbacks and the determined one or more recommendations. For example, as described above with respect to FIGS. 3A and 3B.

Some embodiments may include updating the reference dataset based on the sensors data associated with at least one of the determined one or more feedbacks and the determined one or more recommendations. For example, as described above with respect to FIGS. 3A and 3B.

Some embodiments may include building a second reference dataset based on sensors data collected from multiple devices for supporting a limb and at least one of the determined one or more feedbacks and the determined one or more recommendations associated therewith. For example, as described above with respect to FIGS. 3A and 3B.

Some embodiments may include generating at least one of the one or more feedback and the one or more recommendations based on the second reference dataset. For example, as described above with respect to FIGS. 3A and 3B.

Advantageously, the disclosed device, system and method may provide a solution to a problem of lack of rehabilitation of an injured limb resulting from at least one of a lack of care givers and specifically practitioners, awareness, motivation and access to healthcare services and diagnostics. Provided personalized visual feedbacks and automated diagnosis may enable the users to observe and understand the rehabilitation/healing process and thus may, for example, motivate the users to agree to the healing process and/or to complete the healing process. Provided remote functions may make the diagnostics possible for the users that do not have access to care givers. Provided remote functions may enable care givers who do not have access to healing process monitoring means (e.g., such as X-Ray, EEG, etc.) to monitor the healing process of the users. Affordable, accessible and approachable monitoring of the healing process may significantly improve the health and the lives of people with limb injuries in remote locations, and suitable treatment on time, such as in emergency cases, may help them heal and be a productive, or even save their lives. Furthermore, the device, system and method may provide a platform for integrating many other electronic means to monitor many health conditions, such as detect heart rate and predict falls.

The description below provides some of versions for how the invention may be applied in orthoses.

When wearing a wrist orthosis, the hand is in the optimal position of using the forearm muscles and becomes more functional—measuring motility and muscle tension.

When training the hand with a dynamic orthosis—measure the muscle tension improvement and get a feedback, as well as to alarm about incorrect exercising and how to improve it.

When using an orthosis to fix a fracture, we want to check a good oxygen flow to the soft tissue, that the bone is well fixed and being fused, and that there are no pressure ulcers.

When wearing a rest/cock-up orthosis as a daily rehabilitation practice (especially for people after a stroke/CVA)—detect and produce a feedback of a good blood flow and a positive rehabilitation process of the affected limb.

When wearing a finger orthosis to treat a fracture, it is important to know that the bone is completely fixed, this can be measured by using motion/flex/vibration/force sensors. It is also important to verify there is a good blood flow in the finger, by using the blood flow sensor.

When using a orthosis for neuronal rehabilitation, it is important to have slight vibrations within the orthosis, and for this purpose we may use orthosis that is more thin and flexible. At the same time, we can monitor neural activity by pressure/motion/vibration sensors.

While wearing the orthosis, identification of falls can be detected with accelerometer/magnet/pressure sensors and send alerts to the user's family and care givers. other sensors can detect indicators that can be identify by the ai software and database, that a fall may occur soon and send a warning to the user and its care giver.

Carpal tunnel syndrome—a chronic condition, or a wrist pain, or over exertion of the wrist, is the most common wrist injury, and affects ˜4% of the global population every year. in many cases this condition can be prevented by using the hand in the right position—while typing. the orthosis comprised with sensing unit, can detect wrong patterns and advice of how to correct the position. the same for thumb injuries. for healing, the orthosis comprised with sensing unit, can detect and send positive feedbacks to the user and its caregiver/s.

Heart rate monitoring has multiple proposes, starting from sports which is the most common use, and up to evaluating the user's condition and to alert if the heart rate can't be detected.

Heart rate variability can attest to emotional stress that is unfortunately common among people with disabilities and then alert the care giver. it might also indicate on a severe physical distress and may alert before the user might experience a stroke or a heart attack.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions thereof. The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram portion or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams can represent a module, segment, or portion of code, which includes one or more executable instructions for implementing the specified logical function(s). It should also be noted that, in some alternative implementations, the functions noted in the portion can occur out of the order noted in the figures. For example, two portions shown in succession can, in fact, be executed substantially concurrently, or the portions can sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A device applicable to a limb, the device comprising:
an orthosis comprising one or more holes extending between base surfaces of the orthosis;
one or more sensors, at least one of the one or more sensors is adapted to be attachable to at least one of the one or more holes in the orthosis; and
a data collecting unit connectable to the one or more sensors and configured to receive sensors data from the one or more sensors.

2. The device of claim 1, wherein each of the one or more sensors is selected from a group consisting of: motion sensors, muscle sensors, blood flow sensors, distance sensors, IR Proximity Sensor, force sensors, touch sensors, tilt switch sensors, angle sensors, sound sensors, accelerometer sensors, flex sensors, magnet sensors, RFID sensors, temperature sensors, light sensors, vibration sensors, pressure sensors, ECG sensors, EEG sensors and PPG sensors.

3. The device of claim 1, wherein the orthosis comprises a plate formable into an orthotic member under heating.

4. The device of claim 1, wherein at least one of the one or more sensors is adapted to be placed within at least one of the one or more holes in the orthosis.

5. The device of claim 1, wherein at least one of the one or more sensors comprises:
a sensing unit;
a sensor connector adapted to be placed within at least one of the one or more holes in the orthosis; and
a sensor wire connecting the sensing unit to the sensor connector.

6. The device of claim 5, wherein the sensor connector comprises the data collecting unit.

7. The device of claim 1, further comprising a pad accommodating at least one of the one or more sensors and a sensor wire or the data collecting unit.

8. The device of claim 7, wherein relative locations of the at least one sensor and the data collecting unit in the pad are set to match locations of at least some of the holes in the orthosis.

9. The device of claim 7, wherein the pad comprises one or more pad connectors each adapted to be placed within at least one of the one or more holes in the orthosis.

10. The device of claim 1, further comprising one or more fasteners removably connectable to the orthosis and adapted to fasten the orthosis to the limb.

11. The device of claim 10, wherein at least one of the one or more sensors is attachable to at least one of the one or more fasteners.

12. A system for monitoring a healing processes of a limb, the system comprising:
a device applicable to a limb according to claim 1; and
a computing device connectable to the data collecting unit of the device and configured to receive the sensors data from the data collecting unit thereof.

13. The system of claim 12, wherein the computing device is configured to:
send at least a part of the sensors data to a remote computing device; and
receive, from the remote computing device, at least one of one or more feedbacks and one or more recommendations related to the healing process of the limb.

14. The system of claim 12, wherein the computing device is configured to determine at least one of one or more feedbacks and one or more recommendations related to the healing process of the limb based on at least a part of the sensors data and a reference dataset.

15. The system of claim 13, comprising a user interface, the computing device is configured to present, on the user interface, at least one of the one or more feedbacks and the one or more recommendations.

16. The system of claim 13, comprising a communication unit, the computing device is configured to send, via the communication unit, at least one of the one or more feedbacks and the one or more recommendations to an authorized third party.

17. The system of claim 12, comprising a second computing device configured to:
receive, from the computing device, at least a part of the sensors data;
determine at least one of one or more feedbacks and one or more recommendations related to the healing process of the limb based on the received sensors data and a reference dataset; and
send at least one of the one or more feedbacks and the one or more recommendations to the computing device.

18. The system of claim 17, wherein the device comprises one or more three-dimensional accelerometers, and wherein the second computing device is configured to:
determine, based on readings of the one or more accelerometers, information concerning a motility of the device, and
determine at least one of the one or more feedbacks and the one or more recommendations based on the information concerning the motility of the device.

19. The system of claim 17, wherein the second computing device is configured to:
associate the sensors data with at least one of the determined one or more feedbacks and the determined one or more recommendations; and
update the reference dataset based on the sensors data associated with at least one of the determined one or more feedbacks and the determined one or more recommendations.

20. The system of claim 19, comprising a system database, the second computing device is configured to build a system reference dataset based on sensors data collected from multiple devices for supporting a limb and at least one of the determined one or more feedbacks and the determined one or more recommendations associated therewith.

21.-32. (canceled)
